(19) United States
(12) Patent Application Publication
(10) Pub. No.: US 2009/0143400 A1
(43) Pub. Date: Jun. 4, 2009

(54) PURINE DERIVATIVES HAVING IMMUNO-MODULATING PROPERTIES

(76) Inventors: Thomas McInally, Leicestershire (GB); Stephen Thom, Leicestershire (GB); Hiroki Wada, Leicestershire (GB)

Correspondence Address: MORGAN LEWIS & BOCKIUS LLP, 1111 PENNSYLVANIA AVENUE NW, WASHINGTON, DC, 20004, US

(21) Appl. No.: 12/066,952

(22) PCT Filed: Sep. 12, 2006
(86) PCT No.: PCT/GB2006/003364

§ 371 Date: Aug. 4, 2008

(30) Foreign Application Priority Data
Sep. 16, 2005 (SE) 0502057-3
Sep. 19, 2005 (SE) 0502066-4

(51) Int. Cl. A61K 31/496; C07D 473/18; A61K 31/522; A61P 31/12; A61P 35/00; A61P 37/08; A61P 11/00

(52) U.S. Cl. 514/252.16; 544/276; 514/263.37; 514/263.2

(57) ABSTRACT

The present invention provides compounds of formula

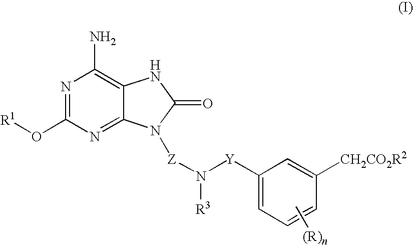

wherein n, Y, Z, R, R¹, R² and R³ are as defined in the specification, processes for their preparation, pharmaceutical compositions containing them and their use in therapy.

The present invention relates to adenine derivatives, processes for their preparation, pharmaceutical compositions containing them and their use in therapy.

The immune system is comprised of innate and acquired immunity, both of which work cooperatively to protect the host from microbial infections. It has been shown that innate immunity can recognize conserved pathogen-associated molecular patterns through toll-like receptors (TLRs) expressed on the cell surface of immune cells. Recognition of invading pathogens then triggers cytokine production (including interferon alpha(IFNα)) and upregulation of co-stimulatory molecules on phagocytes, leading to modulation of T cell function. Thus, innate immunity is closely linked to acquired immunity and can influence the development and regulation of an acquired response.

TLRs are a family of type I transmembrane receptors characterized by an NH₂-terminal extracellular leucine-rich repeat domain (LRR) and a COOH-terminal intracellular tail containing a conserved region called the Toll/IL-1 receptor (TIR) homology domain. The extracellular domain contains a varying number of LRR, which are thought to be involved in ligand binding. Eleven TLRs have been described to date in humans and mice. They differ from each other in ligand specificities, expression patterns, and in the target genes they can induce.

Ligands which act via TLRs (also known as immune response modifiers (IRMS)) have been developed, for example, the imidazoquinoline derivatives described in U.S. Pat. No. 4,689,338 which include the product Imiquimod for treating genital warts, and the adenine derivatives described in WO 98/01448 and WO 99/28321.

International Patent Application No. PCT/JP2005/005401 describes a class of 9-substituted-8-oxoadenine compounds having immuno-modulating properties which act via TLR7 that are useful in the treatment of viral or allergic diseases and cancers.

It has now surprisingly been found that a subset of the compounds generically disclosed in International Patent Application No. PCT/JP2005/005401 possess properties such as increased aqueous solubility which makes them particularly suitable for use in inhalation therapy. Without being bound to any particular theory, it is believed that the increased solubility of the compounds (in the lung) results in increased potency, leading to a reduction in the dose required for efficacy. This in turn improves the safety margins of the compounds.

In accordance with the present invention, there is therefore provided a compound of formula

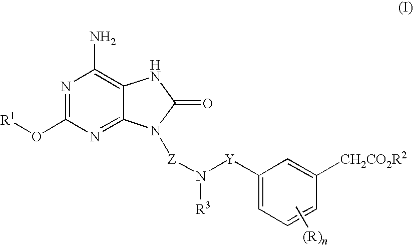

wherein
R¹ represents a C₁-C₆ alkyl group;
Z represents a C₂-C₆ alkylene group;
Y represents a C₁-C₃ alkylene group;
R² represents a C₁-C₆ alkyl group;
n is an integer from 0 to 2;
each group R independently represents halogen, C₁-C₃ alkyl, C₁-C₃ alkoxy or C₁-C₃ haloalkyl;
R³ represents —(CH₂)ₘ—NR⁴R⁵;
m is an integer from 2 to 6;
either R⁴ and R⁵ each independently represent hydrogen or C₁-C₆ alkyl, or R⁴ and R⁵ together with the nitrogen atom to which they are attached form a 3- to 8-membered saturated heterocyclic ring optionally comprising a further ring hetero group NR⁶; and
R⁶ represents hydrogen or C₁-C₆ alkyl;
   or a pharmaceutically acceptable salt thereof.

In the context of the present specification, unless otherwise stated, an alkyl substituent group or an alkyl moiety in a substituent group may be linear or branched. Examples of C₁-C₆ alkyl groups/moieties include methyl, ethyl, n-propyl, isopropyl, n-butyl, isobutyl, tert-butyl, n-pentyl and n-hexyl. Similarly, an alkylene group may be linear or branched. Examples of C₁-C₆ alkylene groups include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene. A C₁-C₃ haloalkyl substituent group will comprise at least one halogen atom, e.g. one, two, three, four or five halogen atoms, examples of which include trifluoromethyl or pentafluoroethyl. When R⁴ and R⁵ together represent a 3- to 8-membered saturated heterocyclic ring, it should be understood that the ring will contain no more than two ring hetero moieties: the nitrogen ring atom to which R⁴ and R⁵ are attached and optionally a group NR⁶.

R¹ represents a C₁-C₆, or C₁-C₅, or C₁-C₄, or C₁-C₃, or C₁-C₂ alkyl group.

In an embodiment of the invention, R¹ represents a C₁-C₄ alkyl group, for example, an n-butyl group.

Z represents a C₂-C₆ alkylene group, for example, a C₂-C₄ or C₃-C₄ alkylene group.

In an embodiment of the invention, Z represents a linear C₂-C₄ or C₃-C₄ alkylene group such as ethylene, n-propylene or n-butylene.

Y represents a C₁-C₃ alkylene group such as methylene, ethylene or n-propylene.

R² represents a C₁-C₆, or C₁-C₅, or C₁-C₄, or C₁-C₃, or C₁-C₂ alkyl group.

In an embodiment of the invention, R² represents a C₁-C₂ alkyl group, for example, a methyl group.

n is an integer 0, 1 or 2.

In an embodiment of the invention, n represents 0.

Each group R independently represents halogen (such as fluorine, chlorine, bromine or iodine), C₁-C₃ alkyl (such as methyl, ethyl or n-propyl), C₁-C₃ alkoxy (such as methoxy, ethoxy or n-propoxy), or C₁-C₃ haloalkyl (such as dibromomethyl, dichloromethyl, bromochloromethyl, trifluoromethyl or pentafluoroethyl).

In an embodiment of the invention, n is 1 or 2 and each group R represents a halogen atom, e.g. fluorine.

R³ represents —(CH₂)ₘ—NR⁴R⁵ where m is an integer 2, 3, 4, 5 or 6.

In an embodiment of the invention, m is 2 or 3.

In an embodiment of the invention, R⁴ and R⁵ each independently represent hydrogen or C₁-C₆, or C₁-C₅, or C₁-C₄, or C₁-C₃, or C₁-C₂ alkyl.

In one embodiment, R⁴ and R⁵ each independently represent hydrogen or methyl.

In an alternative embodiment, R⁴ and R⁵ together with the nitrogen atom to which they are attached form a 3-, 4-, 5-, 6-, 7- or 8-membered, e.g. 5- to 6-membered, saturated heterocyclic ring optionally comprising a further ring hetero group NR⁶ where R⁶ represents hydrogen or C₁-C₆, or C₁-C₅, or C₁-C₄, or C₁-C₃, or C₁-C₂ alkyl. Examples of heterocyclic rings include azetidinyl, pyrrolidinyl, piperidinyl, piperazinyl and 4-methylpiperazin-1-yl.

In an embodiment of the invention,
R¹ represents n-butyl;
Z represents n-propylene (CH₂)₃) or n-butylene ((CH₂)₄);
Y represents methylene;
R² represents methyl;
n is O;
R³ represents —(CH₂)ₘ—NR⁴R⁵;
m is 2 or 3;
either R⁴ and R⁵ each independently represent hydrogen or methyl, or R⁴ and R⁵ together with the nitrogen atom to which they are attached form a 5- to 6-membered saturated heterocyclic ring optionally comprising a further ring hetero group NR⁶; and
R⁶ represents methyl.

Examples of compounds of the invention include
Methyl [3-({[3-(6-amino-2-butoxy-8-oxo-7,8-dihydro-9H-purin-9-yl)propyl][3-(dimethylamino)propyl]amino}methyl)phenyl]acetate,
Methyl (3-{[[3-(6-amino-2-butoxy-8-oxo-7,8-dihydro-9H-purin-9-yl)propyl](3-pyrrolidin-1-ylpropyl)amino]methyl}phenyl)acetate,
Methyl [3-({[3-(6-amino-2-butoxy-8-oxo-7,8-dihydro-9H-purin-9-yl)propyl][3-(4-methylpiperazin-1-yl)propyl]amino}methyl)phenyl]acetate,
Methyl [3-({[3-(6-amino-2-butoxy-8-oxo-7,8-dihydro-9H-purin-9-yl)propyl][2-(dimethylamino)ethyl]amino}methyl)phenyl]acetate,
Methyl [3-({[3-(6-amino-2-butoxy-8-oxo-7,8-dihydro-9H-purin-9-yl)propyl][3-(methylamino)propyl]amino}methyl)phenyl]acetate,
Methyl [3-({[4-(6-amino-2-butoxy-8-oxo-7,8-dihydro-9H-purin-9-yl)butyl][3-(4-methylpiperazin-1-yl)propyl]amino}methyl)phenyl]acetate,
   and pharmaceutically acceptable salts of any one thereof.

The present invention further provides a process for the preparation of a compound of formula (I) or a pharmaceutically acceptable salt thereof as defined above which comprises,

(a) reacting a compound of formula

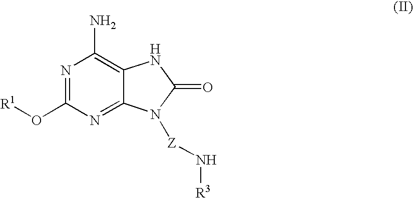

wherein Z, R¹ and R³ are as defined in formula (I), with a compound of formula

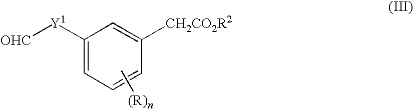

wherein Y¹ represents a bond or C₁-C₂ alkylene group and n, R and R² are as defined in formula (I) in the presence of a suitable reducing agent (e.g. sodium triacetoxyborohydride); or
(b) reacting a compound of formula (II) as defined in (a) above with a compound of formula

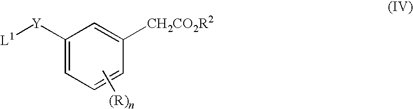

wherein L¹ represents a leaving group (e.g. halogen, mesylate or triflate) and n, Y, R and R² are as defined in formula (I) in the presence of a suitable base (e.g. sodium carbonate or potassium carbonate); or
(c) reacting a compound of formula

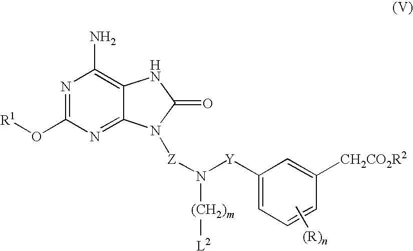

wherein L² represents a leaving group (e.g. halogen, mesylate or triflate) and m, n, Y, Z, R, R¹ and R² are as defined in formula (I), with a compound of formula (VI), HNR⁴R⁵, wherein R⁴ and R⁵ are as defined in formula (I); or
(d) reacting a compound of formula

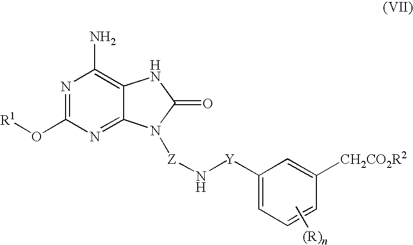

wherein n, Y, Z, R, R¹ and R² are as defined in formula (I), with a compound of formula

L³-(CH₂)ₘ—NR⁴R⁵ (VIII)

wherein L³ represents a leaving group (e.g. halogen, mesylate or triflate) and m, R⁴ and R⁵ are as defined in formula (I); or
(e) reacting a compound of formula (VII) as defined in (d) above with a compound of formula

OHC—(CH₂)ₘ₋₁—NR⁴R⁵ (IX)

wherein m, R⁴ and R⁵ are as defined in formula (I) in the presence of a suitable reducing agent (e.g. sodium triacetoxyborohydride);
and optionally after (a), (b), (c), (d) or (e) carrying out one or more of the following:
converting the compound obtained to a further compound of the invention
forming a pharmaceutically acceptable salt of the compound.

In process (a), the reaction may conveniently be carried out in an organic solvent such as 1-methyl-2-pyrrolidinone, 1,2-dichloroethane or tetrahydrofuran at a temperature, for example, in the range from 0 to 150° C.

In process (b), the reaction may conveniently be carried out in an organic solvent such as acetonitrile, 1-methyl-2-pyrrolidinone or N,N-dimethylformamide at a temperature, for example, in the range from 0 to 150° C.

In process (c), the reaction may conveniently be carried out in an organic solvent such as acetonitrile, 1-methyl-2-pyrrolidinone or N,N-dimethylformamide at a temperature, for example, in the range from 0 to 150° C.

In process (d), the reaction may conveniently be carried out in an organic solvent such as 1-methyl-2-pyrrolidinone or N,N-dimethylformamide at a temperature, for example, in the range from 0 to 150° C.

In process (e), the reaction may conveniently be carried out in an organic solvent such as 1-methyl-2-pyrrolidinone, 1,2-dichloroethane or tetrahydrofuran at a temperature, for example, in the range from 0 to 150° C.

Compounds of formula (II) may be prepared as illustrated in the following reaction scheme:

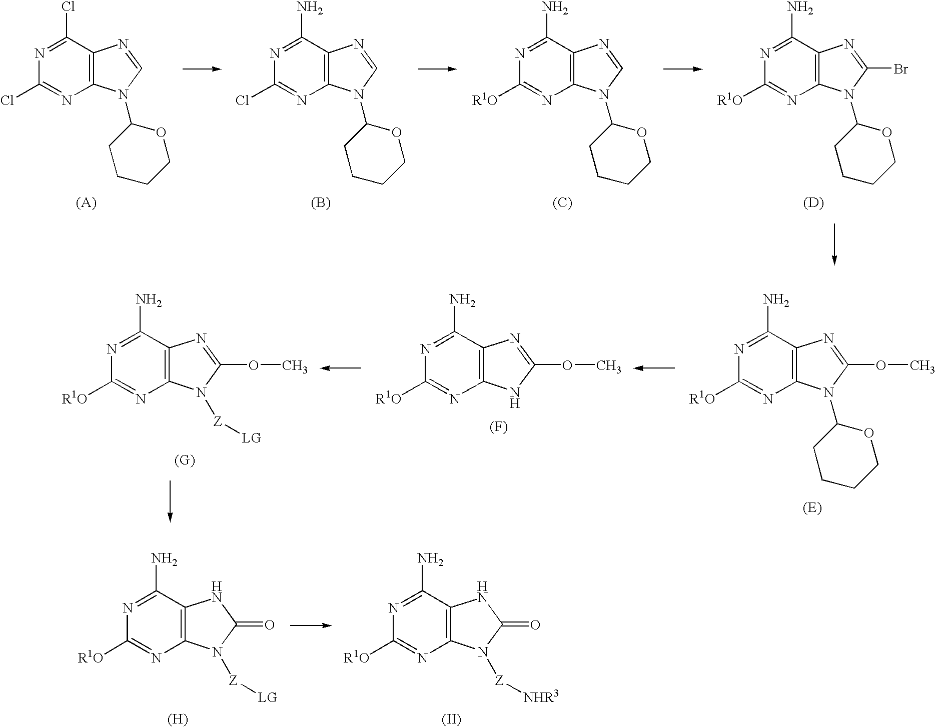

The compound of formula (B) is prepared by reacting the compound of formula (A) with ammonia in an organic solvent such as methanol, ethanol, propanol, butanol, tetrahydrofuran, 1,4-dioxane, diglyme, acetonitrile or an aqueous mixture of any one of the preceding solvents. The reaction may be carried out in an autoclave, and at a temperature, for example, in the range from 20 to 200° C.

Compounds of formula (C) may be prepared by reacting the compound of formula (B) with a C₁-C₆ alkanol in the presence of a base such as sodium hydride and in an organic solvent such as tetrahydrofuran, 1,4-dioxane, diglyme, N,N-dimethylformamide or dimethylsulfoxide, preferably at elevated temperature, e.g. at a temperature in the range from 20 to 150° C. Alternatively an alkali metal such as sodium may be dissolved in the C₁-C₆ alkanol and then reacted with the compound of formula (B), preferably at elevated temperature, e.g. at a temperature in the range from 20 to 150° C.

Compounds of formula (D) are prepared by brominating a compound of formula (C). The reaction may be carried out using a brominating agent such as bromine, hydroperbromic acid or N-bromosuccinimide, in an organic solvent such as carbon tetrachloride, methylene chloride, dichloroethane, diethyl ether, acetic acid or carbon disulfide. The reaction temperature will generally be in the range from 0° C. to the boiling point of the solvent.

Compounds of formula (E) are prepared by reacting a compound of formula (D) with sodium methoxide in an organic solvent such as methanol and at a temperature, for example, in the range from 20 to 150° C.

Compounds of formula (F) may be obtained by treating a compound of formula (E) with an acid such as trifluoroacetic acid in an organic solvent such as methanol.

Compounds of formula (G) are prepared by reacting a compound of formula (F) with a compound of formula LG-Z-LG wherein LG represents a leaving group such as a halogen, mesylate or triflate and Z represents a C₂-C₆ alkylene group as defined in formula (II). The reaction may be carried out in an organic solvent such as N,N-dimethylformamide, dimethylsulfoxide or acetonitrile with a base present, preferably at room temperature (20° C.). A base such as an alkali metal carbonate, e.g. sodium carbonate or potassium carbonate; an alkaline earth metal carbonate, e.g. calcium carbonate; a metal hydroxide, e.g. sodium hydroxide or potassium hydroxide; a metal hydrogenate, e.g. sodium hydride; or a metal alkoxide, e.g. potassium t-butoxide, may be used.

Compounds of formula (H) may be obtained by treatment of a compound of formula (G) with an acid. The reaction may be carried out in an organic solvent such as methanol using either an inorganic acid such as hydrochloric acid, hydrobromic acid or sulfuric acid, or an organic acid such as trifluoroacetic acid.

Compounds of formula (II) are prepared by reacting a compound of formula (H) with an amine of formula R³NH₂ where R³ is as defined in formula (II). The reaction may be carried out in an organic solvent such as acetonitrile or N,N-dimethylformamide using an excess of the amine, preferably at elevated temperature, e.g. at a temperature in the range from 0 to 150° C.

Compounds of formula (V) may be prepared by reacting a compound of formula

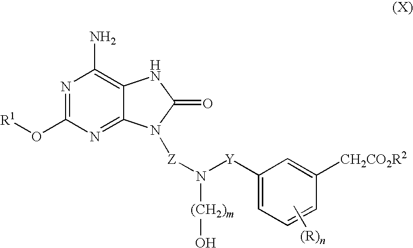

wherein m, n, Y, Z, R, R¹ and R² are as defined in formula (V) using standard procedures. For example, compounds of formula (V) in which L² represents halogen, e.g. chlorine, may be prepared by reaction with a halogenating reagent such as thionyl chloride in an organic solvent such as dichloromethane at room temperature (20° C.).

Compounds of formula (X) may be prepared by reacting a compound of formula

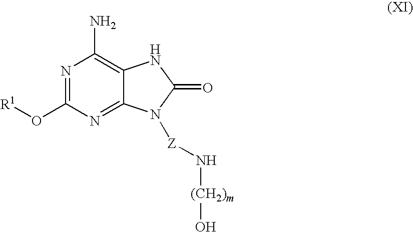

wherein m, Z and R¹ are as defined in formula (x), with a compound of formula (III) or (IV) as defined above and under the same conditions as described in processes (a) and (b) respectively.

Compounds of formula (XI) may be prepared by reacting a compound of formula (H) as defined above with a C₂-C₆ aminoalcohol in an organic solvent such as acetonitrile or N,N-dimethylformamide using an excess of the aminoalcohol, preferably at elevated temperature, e.g. at a temperature in the range from 20 to 150° C.

Compounds of formula (VII) may be prepared by processes analogous to those described for the preparation of compounds of formula (X) using a compound of formula

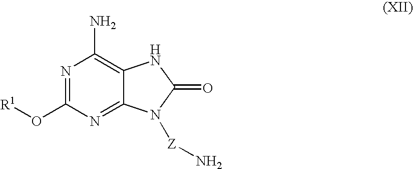

wherein Z and R¹ are as defined in formula (VII).

Compounds of formula (XII) may be obtained by reacting a compound of formula (F) as defined above with a compound of formula (XIII), L⁴-Z-NH—P, wherein L⁴ represents a leaving group (e.g. halogen, mesylate or triflate), P represents a nitrogen-protecting group (e.g. butoxycarbonyl) and Z is as defined in formula (XII), followed by removal of the nitrogen-protecting group, P, and removal of the oxygen-protecting group in the substituent —OCH₃.

The reaction between the compounds of formula (F) and (XIII) may be carried out in an organic solvent such as N,N-dimethylformamide, dimethylsulfoxide or acetonitrile with a base present, at a temperature, for example, in the range from 0 to 150° C. The base used may be an alkali metal carbonate, e.g. sodium carbonate or potassium carbonate; an alkaline earth metal carbonate, e.g. calcium carbonate; a metal hydroxide, e.g. sodium hydroxide or potassium hydroxide; a metal hydrogenate, e.g. sodium hydride; or a metal alkoxide, e.g. potassium tert-butoxide. The removal of the protecting groups may be carried out according to methods known in the art.

Compounds of formulae (III), (IV), (VI), (VIII), (IX) and (XIII) are either commercially available, are known in the literature or may be prepared using known techniques.

Compounds of formula (I) can be converted into further compounds of formula (I) using standard procedures. For example a compound of formula (I) where R²=methyl can be converted to a compound of formula (I) where R²=ethyl by treatment with a solution of hydrogen chloride in ethanol, at a temperature, for example in the range from 20 to 78° C.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, preferably an acid addition salt such as a hydrochloride, hydrobromide, trifluoroacetate, sulphate, phosphate, acetate, fumarate, maleate, tartrate, lactate, citrate, pyruvate, succinate, oxalate, methanesulphonate orp-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as modulators of toll-like receptor (especially TLR7) activity, and thus may be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
3. eyes: blepharitis; conjunctivitis, including perennial and vernal allergic conjunctivitis; iritis; anterior and posterior uveitis; choroiditis; autoimmune, degenerative or inflammatory disorders affecting the retina; ophthalmitis including sympathetic ophthalmitis; sarcoidosis; infections including viral, fungal, and bacterial;
4. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
5. allograft rejection: acute and chronic following, for example, transplantation of kidney, heart, liver, lung, bone marrow, skin or cornea or following blood transfusion; or chronic graft versus host disease;
6. other auto-immune and allergic disorders including rheumatoid arthritis, irritable bowel syndrome, systemic lupus erythematosus, multiple sclerosis, Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome and Sazary syndrome;
7. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
8. infectious diseases: virus diseases such as genital warts, common warts, plantar warts, hepatitis B, hepatitis C, herpes simplex virus, molluscum contagiosum, variola, human immunodeficiency virus (HIV), human papilloma virus (HPV), cytomegalovirus (CMV), varicella zoster virus (VZV), rhinovirus, adenovirus, coronavirus, influenza, para-influenza; bacterial diseases such as tuberculosis and mycobacterium avium, leprosy; other infectious diseases, such as fungal diseases, chlamydia, candida, aspergillus, cryptococcal meningitis, pneumocystis carnii, cryptosporidiosis, histoplasmosis, toxoplasmosis, trypanosome infection and leishmaniasis.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In particular, the compounds of the invention may be used in the treatment of asthma, COPD, allergic rhinitis, allergic conjunctivitis, atopic dermatitis, cancer, hepatitis B, hepatitis C, HIV, HPV, bacterial infections and dermatosis.

The invention still further provides a method of treating, or reducing the risk of, an obstructive airways disease or condition (e.g. asthma or COPD) which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, is of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention (including pharmaceutically acceptable salts) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention or a pharmaceutical composition or formulation comprising a compound of the invention is administered concurrently or sequentially or as a combined preparation with another therapeutic agent or agents, for the treatment of one or more of the conditions listed.

In particular, for the treatment of the inflammatory diseases COPD, asthma and allergic rhinitis the compounds of the invention may be combined with agents such as tumour necrosis factor alpha (TNF-alpha) inhibitors such as anti-TNF monoclonal antibodies (for example Remicade, CDP-870 and adalimumab) and TNF receptor immunoglobulin molecules (such as Enbrel); non-selective cyclo-oxygenase COX-1/COX-2 inhibitors whether applied topically or systemically (such as piroxicam, diclofenac, propionic acids such as naproxen, flubiprofen, fenoprofen, ketoprofen and ibuprofen, fenamates such as mefenamic acid, indomethacin, sulindac, azapropazone, pyrazolones such as phenylbutazone, salicylates such as aspirin), COX-2 inhibitors (such as meloxicam, celecoxib, rofecoxib, valdecoxib, lumarocoxib, parecoxib and etoricoxib); glucocorticosteroids (whether administered by topical, oral, intramuscular, intravenous, or intra-articular routes); methotrexate, lefunomide; hydroxychloroquine, d-penicillamine, auranofin or other parenteral or oral gold preparations.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT B4, LTC4, LTD4, and LTE4) selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention and an anticholinergic agent including muscarinic receptor (M1, M2, and M3) antagonists such as atropine, hyoscine, glycopyrrrolate, ipratropium bromide, tiotropium bromide, oxitropium bronide, pirenzepine or telenzepine.

The present invention still further relates to the combination of a compound of the invention together with a beta-adrenoceptor agonist (including beta receptor subtypes 1-4) such as isoprenaline, salbutamol, formoterol, salmeterol, terbutaline, orciprenaline, bitolterol mesylate, and pirbuterol.

The present invention further relates to the combination of a compound of the invention and a chromone, such as sodium cromoglycate or nedocromil sodium.

The present invention still further relates to the combination of a compound of the invention together with an insulin-like growth factor type I (IGF-1) mimetic.

The present invention still further relates to the combination of a compound of the invention and a glucocorticoid, such as flunisolide, triamcinolone acetonide, beclomethasone dipropionate, budesonide, fluticasone propionate, ciclesonide or mometasone furoate.

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12.

The present invention still further relates to the combination of a compound of the invention together with modulators of chemokine receptor function such as antagonists of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX3CR1 for the C—X3-C family.

The present invention still further relates to the combination of a compound of the invention together with a cytokine or modulator of cytokine function, including alpha-, beta-, and gamma-interferon; interleukins (IL) including IL1 to 15, and interleukin antagonists or inhibitors, including agents which act on cytokine signalling pathways.

The present invention still further relates to the combination of a compound of the invention together with an immunoglobulin (Ig) or Ig preparation or an antagonist or antibody modulating Ig function such as anti-IgE (omalizumab).

The present invention further relates to the combination of a compound of the invention and another systemic or topically-applied anti-inflammatory agent, such as thalidomide or a derivative thereof, a retinoid, dithranol or calcipotriol.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

A compound of the invention can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will be further explained by reference to the following illustrative examples.

Unless otherwise stated organic solutions were dried over magnesium sulphate. RPHPLC denotes Reversed Phase preparative High Performance Liquid Chromatography using Waters Symmetry C8, Xterra or Phenomenex Gemini columns using acetonitrile and either aqueous amionium acetate, ammonia, formic acid or trifluoroacetic acid as buffer where appropriate. Column chromatography was carried out on silica gel. SCX denotes solid phase extraction with a sulfonic acid sorbent whereby a mixture was absorbed on a sulfonic acid sorbent and eluted with an appropriate solvent such as methanol or acetonitrile and then the free base product was eluted with aqueous ammonia/methanol or acetonitrile.

### EXAMPLE 1

### Methyl [3-({[3-(6-amino-2-butoxy-8-oxo-7,8-dihydro-9H-purin-9-yl)propyl][3-(dimethylamino)propyl]amino}methyl)phenyl]acetate

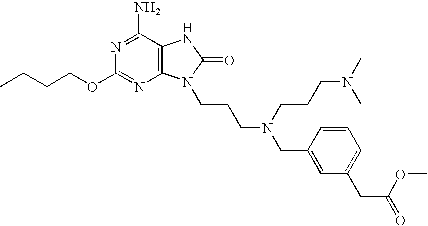

### (i) 2-Chloro-9-(tetrahydro-2H-pyran-2-yl)-9H-purin-6-amine

2,6-Dichloro-9-(tetrahydro-2H-pyran-2-yl)-9H-purine (55 g) was dissolved in 7N-aqueous ammonia in methanol (500 ml) and heated at 100° C. in a sealed flask for 6 hours. The reaction mixture was cooled to room temperature and left overnight. Filtration afforded the subtitle compound. Yield 40 g.

¹H NMR δ (CDCl₃) 8.02 (1H, s), 5.94 (2H, brs), 5.71 (1H, dd), 4.15-4.22 (1H, m), 3.75-3.82 (1H, m), 1.27-2.12 (6H, m).

### (ii) 2-Butoxy-9-(tetrahydro-2H-pyran-2-yl)-9H-purin-6-amine

The product from step (i) (40 g) was dissolved in 19% (w/w)-sodium butoxide in butanol (250 ml). The reaction mixture was stirred under reflux for 6 hours. The resultant suspension was cooled to room temperature, diluted with water (250 ml) and extracted with diethyl ether (200 ml×3). The combined organic phase was washed with water (200 ml×3), dried and concentrated in vacuo. The subtitle compound was crystallised from diethyl ether/isohexane (1/1, 300 ml) and obtained by filtration. Yield 19 g.

¹H NMR δ (CDCl₃) 7.87 (1H, s), 5.56-5.68 (3H, m), 4.31-4.35 (2H, t), 4.14-4.17 (1H, m), 3.76-3.80 (1H, m), 1.49-2.08 (10H, m), 0.98 (3H, t).

### (iii) 8-Bromo-2-butoxy-9-(tetrahydro-2H-pyran-2-yl) 9H-purin-6-amine

The product from step (ii) (30 g) was dissolved in dry dichloromethane (200 ml). The solution was stirred at room temperature whilst N-bromosuccinamide (27 g) was added portionwise. The mixture was stirred at ambient temperature overnight. 20% (w/v)-Sodium sulfate (200 ml) was added and the separated aqueous phase extracted with dichloromethane (200 ml×3). The combined organic phase was washed with saturated sodium hydrogen carbonate solution (200 ml×2) and brine (200 ml). After concentration in vacuo, the residue was dissolved in ethyl acetate (300 ml), washed with water (200 ml), brine (200 ml) and dried. The solution was filtered through silica gel and concentrated in vacuo. The residue was triturated with diethyl ether and isohexane (1/1, 200 ml) then filtered to give the subtitle compound (26 g). The filtrate was concentrated in vacuo and the residue was purified by column chromatography (ethyl acetate/isohexane) to give a further 2.5 g of product. The solids were combined to give the subtitle compound as a yellow solid. Yield 28.5 g. Melting point: 148-150° C.

¹H NMR δ (CDCl₃) 5.59-5.64 (3H, m), 4.32 (2H, m), 4.17 (1H, m), 3.74 (1H, m), 3.08 (1H, m), 2.13 (1H, d), 1.48-1.83 (8H, m), 0.98 (3H, t).

### (iv) 2-Butoxy-8-methoxy-9-(tetrahydro-2H-pyran-2-yl) 9H-purin-6-amine

Sodium (3.7 g) was added to absolute methanol (400 ml) under a nitrogen atmosphere. To this solution was added the product from step (iii) (28.5 g) and the mixture was stirred at 65° C. for 9 hours. The mixture was concentrated in vacuo and 500 ml of water added. The aqueous phase was extracted with ethyl acetate (300 ml×2), washed with brine (200 ml×3) and dried. The subtitle compound was obtained after crystallisation from diethyl ether. Yield 14.2 g.

¹H NMR δ (CDCl₃) 5.51 (1H, dd), 5.28 (2H, brs), 4.29 (2H, t), 4.11-4.14 (4H, m), 3.70 (1H, m), 2.76-2.80 (1H, m), 2.05 (1H, d), 1.47-1.81 (8H, m), 0.97 (3H, t).

### (v) 2-Butoxy-8-methoxy-9H-purin-6-amine, trifluoroacetate salt

The product from step (iv) (24 g) was dissolved in absolute methanol (300 ml) and 30 ml of trifluoroacetic acid was added. The reaction mixture was stirred at ambient temperature for 3 days and concentrated in vacuo. The subtitle compound was obtained as a white crystalline solid after trituration with methanol/ethyl acetate. Yield 21 g.

¹H NMR δ (CD₃OD) 4.48 (2H, t), 4.15 (3H, s), 1.80 (2H, quintet), 1.50 (2H, sextet), 0.99 (3H, t).

### (vi) 9-(3-Bromopropyl)-2-butoxy-8-methoxy-9H-purin-6-amine

The product of step (v) (20 g) was added in portions over 10 minutes to a rapidly stirred mixture of potassium carbonate (40 g) and 1,3-dibromopropane (34 ml) in N,N-dimethylformamide (250 ml) at ambient temperature and the mixture stirred for 1.5 hours. The mixture was diluted with water (800 ml) and extracted with ethyl acetate (300 ml×3). The combined extracts were washed with brine (200 ml) and dried. The mixture was purified by column chromatography (ethyl acetate), to afford the subtitle compound as a white solid. Yield 16 g.

¹H NMR δ (CDCl₃) 5.19 (2H, s), 4.28 (2H, J=6.7 Hz, t), 4.12 (3H, s), 4.09 (2H, J=9.4 Hz, t), 3.37 (2H, J=13.3 Hz, t), 2.39-2.30 (2H, m), 1.81-1.72 (2H, m), 1.55-1.43 (2H, m), 0.96 (3H, J=11.4 Hz, t).

### (vii) 6-Amino-9-(3-bromopropyl)-2-butoxy-7,9-dihydro-8H-purin-8-one

The product of step (vi) (35.8 g) was dissolved in methanol (400 ml) and treated with 4M hydrogen chloride in dioxane (100 ml). The mixture was stirred at ambient temperature for 6 hours and concentrated in vacuo. Dichloromethane (500 ml) was added and concentrated in vacuo, which afforded a foam that was taken onto the next step without further purification. Yield 38 g.

¹H NMR δ (DMSO-d₆) 10.60 (1H, s), 4.45 (2H, m), 3.84 (2H, m), 3.65 (2H, m), 2.19 (2H, m), 1.66-1.73 (2H, m), 1.36-1.47 (2H, m), 0.96 (3H, m).

### (viii) 6-Amino-2-butoxy-9-{3-[(3-hydroxypropyl)amino]propyl}-7,9-dihydro-8H-purin-8-one

The product of step (vii) (6 g) was suspended in acetonitrile (100 ml) and 3-aminopropan-1-ol (20 ml) was added. The mixture was stirred under reflux overnight. After cooling to room temperature, the mixture was concentrated in vacuo and 20% (w/v)-aqueous sodium hydrogen carbonate was added (100 ml). The suspension was stirred at ambient temperature overnight, the solid collected via filtration, stored under high vacuum for 16 hours and dried to give the subtitle compound as a white solid. Yield 4.75 g.

¹H NMR δ (DMSO-d₆) 6.40 (2H, brs), 4.15 (2H, J=6.6 Hz, t), 3.70 (2H, J=6.9 Hz, t), 3.44 (2H, m), 2.60-2.27 (4H, m), 1.77-1.23 (8H, m), 0.92 (3H, J=7.5 Hz, t).

MS: APCI (+ve): 339 (M+H)

### (ix) Methyl (3-{[[3-(6-amino-2-butoxy-8-oxo-7,8-dihydro-9H-purin-9-yl)propyl](3-hydroxypropyl)amino]methyl}phenyl)acetate

The product of step (viii) (4.75 g) was dissolved in N,N-dimethylformamide (40 ml). Potassium carbonate (2.00 g) and methyl [3-(bromomethyl)phenyl]acetate (3.56 g) were added. The mixture was stirred at ambient temperature overnight. 20% (w/v) aqueous sodium hydrogen carbonate was added (20 ml) and the suspension stirred at ambient temperature overnight. The solid was collected via filtration, dried under high vacuum for 16 hours to give the subtitle compound as a white solid. Yield 4.91 g.

¹H NMR δ (DMSO-d₆) 7.25-7.09 (4H, m), 6.39 (2H, brs), 4.33 (1H, J=4.8 Hz, t), 4.12 (2H, J=6.6 Hz, t), 3.66 (2H, J=7.2 Hz, t), 3.64 (2H, s), 3.60 (3H, s), 3.50 (2H, s), 3.42-3.65 (2H, m), 2.44-2.27 (4H, m), 1.83-1.31 (8H, m), 0.90 (3H, J=7.2 Hz, t).

MS: APCI (+ve): 501 (M+H)

### (x) Methyl [3-({[3-(6-amino-2-butoxy-8-oxo-7,8-dihydro-9H-purin-9-yl)propyl][3-(dimethylamino)propyl]amino}methyl)phenyl]acetate

The product of step (ix) (200 mg) was suspended in dichloromethane (3 ml) and thionyl chloride (0.06 ml) added. The solution was stirred at ambient temperature for 5 hours and concentrated in vacuo azeotropically with toluene (100 ml). Sodium iodide (200 mg) and 2M solution of N,N-dimethylamine (4 ml) in tetrahydrofuran were added. The mixture was heated at 50° C. for 72 hours in a sealed tube. After cooling to ambient temperature, the mixture was treated with SCX and purified by RPHPLC, to afford the title compound as a white solid. Yield 96 mg.

¹H NMR δ (DMSO-d₆) 7.25-7.08 (4H, m), 6.52 (2H, brs), 4.11 (1H, J=6.6 Hz, t), 3.66 (2H, J=6.9 Hz, t), 3.63 (2H, s), 3.59 (3H, s), 3.49 (2H, s), 2.43-2.32 (4H, m), 2.12 (2H, J=6.9 Hz, t), 2.03 (6H, s), 1.66-1.30 (8H, m), 0.90 (3H, J=7.2 Hz, t).

MS: APCI (+ve): 528 (M+H)

### EXAMPLE 2

### Methyl (3-{[[3-(6-amino-2-butoxy-8-oxo-7,8-dihydro-9H-purin-9-yl)propyl](3-pyrrolidin-1-ylpropyl)amino]methyl}phenyl)acetate

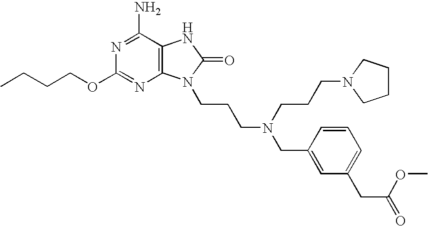

The title compound was prepared by a method analogous to that described in Example 1 above using pyrrolidine. Yield 150 mg.

¹H NMR δ (DMSO-d₆) 7.25-7.09 (4H, m), 6.37 (2H, brs), 4.12 (1H, J=6.6 Hz, t), 3.66 (2H, J=7.2 Hz, t), 3.64 (2H, s), 3.59 (3H, s), 3.49 (2H, s), 3.31-2.72 (2H, m), 2.72-2.29 (10H, m), 1.82-1.23 (10H, m), 0.90 (3H, J=7.2 Hz, t).

MS: APCI (+ve): 554 (M+H)

### EXAMPLE 3

### Methyl [3-({[3-(6-amino-2-butoxy-8-oxo-7,8-dihydro-9H-purin-9-yl)propyl][3-(4-methylpiperazin-1-yl)propyl]amino}methyl)phenyl]acetate

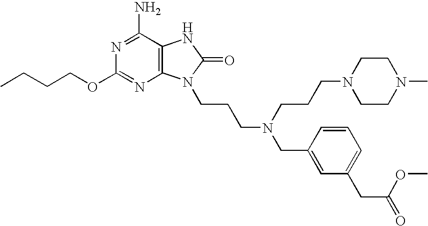

The title compound was prepared by a method analogous to that described in Example 1 above using N-methylpiperazine. Yield 50 mg.

¹H NMR δ (DMSO-d₆) 7.24-7.09 (4H, m), 6.45 (2H, brs), 4.11 (1H, J=6.4 Hz, t), 3.66 (2H, J=7.2 Hz, t), 3.63 (2H, s), 3.59 (3H, s), 3.48 (2H, s), 3.31 (2H, m), 2.49-2.16 (12H, is m), 2.10 (3H, s), 1.83-1.24 (8H, m), 0.90 (3H, J=7.2 Hz, t).

MS: APCI (+ve): 583 (M+H)

### EXAMPLE 4

### Methyl [3-({[3-(6-amino-2-butoxy-8-oxo-7,8-dihydro-9H-purin-9-yl)propyl][2-(dimethylamino)ethyl]amino}methyl)phenyl]acetate

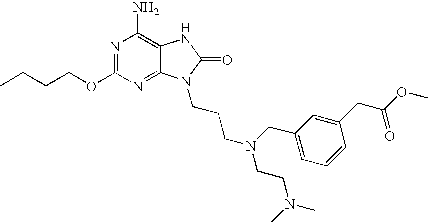

### (i) 6-Amino-2-butoxy-9-(3-{[2-(dimethylamino)ethyl]amino}propyl)-7,9-dihydro-8H-purin-8-one

The subtitle compound was prepared by a method analogous to that of Example 1 step (viii) using N,N-dimethylethane-1,2-diamine. Yield 0.5 g.

MS: APCI (+ve): 352 (M+1)

### (ii) Methyl [3-({[3-(6-amino-2-butoxy-8-oxo-7,8-dihydro-9H-purin-9-yl)propyl][2-(dimethylamino)ethyl]amino}methyl)phenyl]acetate

The product from step (i) (500 mg) was dissolved in a mixture of 1,2-dichloroethane (12 ml) and 1-methyl-2-pyrrolidinone (3 ml). (3-Formyl-phenyl)-acetic acid methyl ester (300 mg) and sodium triacetoxyborohydride (425 mg) were added and the mixture stirred at ambient temperature for 4 hours. After removing the solvent, the residue was partitioned between dichloromethane (100 ml) and saturated aqueous sodium hydrogen carbonate (100 ml), the organic layer dried and concentrated in vacuo. The residue was purified by RPHPLC to afford the title compound. Yield 260 mg.

¹H NMR δ (DMSO-d₆) 9.82 (1H, s), 7.24-7.16 (3H, m), 7.10 (1H, d), 6.38 (2H, s), 4.12 (2H, t), 3.68 (2H, t), 3.64 (2H, s), 3.59 (3H, s), 3.52 (2H, s), 2.45-2.41 (4H, m), 2.27-2.23 (2H, m), 2.04 (6H, s), 1.85-1.80 (2H, m), 1.65-1.58 (2H, m), 1.39-1.33 (2H, m), 0.90 (3H, t).

MS: APCI (+ve): 514

### EXAMPLE 5

### Methyl [3-({[3-(6-amino-2-butoxy-8-oxo-7,8-dihydro-9H-purin-9-yl)propyl][3-(methylamino)propyl]amino}methyl)phenyl]acetate

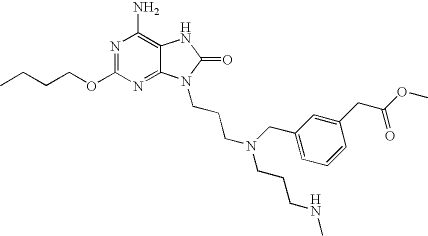

### (i) tert-Butyl (3-{[3-(6-amino-2-butoxy-8-oxo-7,8-dihydro-9H-purin-9-yl)propyl]amino}propyl)methylcarbamate

The subtitle compound was prepared by a method analogous to that of Example 1 step (viii) using tert-butyl (3-aminopropyl)methylcarbamate. Yield 430 mg.

MS: APCI (+ve): 452

### (ii) Methyl {3-[([3-(6-amino-2-butoxy-8-oxo-7,8-dihydro-9H-purin-9-yl)propyl]{3-[(tert-butoxycarbonyl)(methyl)amino]propyl}amino)methyl]phenyl}acetate

The subtitle compound was prepared by a method analogous to that of Example 1 step (ix) using the product of step (i). The subtitle compound obtained (200 mg) was taken onto the next step without further purification.

MS: APCI (+ve): 614

### (iii) Methyl [3-({[3-(6-amino-2-butoxy-8-oxo-7,8-dihydro-9H-purin-9-yl)propyl][3-(methylamino)propyl]amino}methyl)phenyl]acetate

The product from step (ii) (200 mg) was dissolved in methanol (5 ml) and 4M hydrogen chloride in dioxane (5 ml) added. The mixture was stirred at room temperature for 72 hours and concentrated in vacuo. The mixture was purified by RPHPLC, to afford the title compound as a white solid. Yield 35 mg.

¹H NMR δ (DMSO-d₆) 7.24-7.15 (3H, m), 7.10 (1H, d), 6.40 (2H, s), 4.12 (2H, t), 3.67 (2H, t), 3.64 (2H, s), 3.59 (3H, s), 3.48 (2H, s), 2.42-2.36 (6H, m), 2.20 (3H, s), 1.85-1.78 (2H, m), 1.65-1.58 (2H, m), 1.54-1.47 (2H, m), 1.41-1.31 (2H, m), 0.90 (3H, t).

MS: APCI (+ve): 514

### EXAMPLE 6

### Methyl [3-({[4-(6-amino-2-butoxy-8-oxo-7,8-dihydro-9H-purin-9-yl)butyl][3-(4-methylpiperazin-1-yl)propyl]amino}methyl)phenyl]acetate

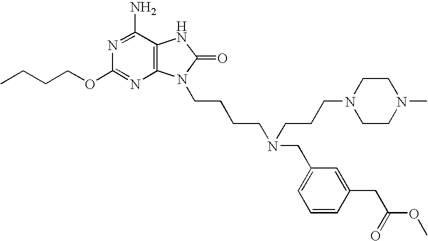

### (i) 9-(3-Bromopropyl)-2-butoxy-8-methoxy-9H-purin-6-amine

The subtitle compound was prepared by a method analogous to that of Example 1 step (vi) using 1,4-dibromobutane. Yield 16 g.

MS: APCI (+ve): 373/375=1/1 (M+H) bromide isotope pattern

### (ii) 3-{[4-(6-Amino-2-butoxy-8-methoxy-9H-purin-9-yl)butyl]amino}propan-1-ol

The subtitle compound was prepared by a method analogous to that of Example 1 step (viii) using the product of step (i). Yield 6 g.

MS: APCI (+ve): 339 (4+H)

### (iii) Methyl (3-{[[4-(6-amino-2-butoxy-8-methoxy-9H-purin-9-yl)butyl](3-hydroxypropyl)amino]methyl}phenyl)acetate

The subtitle compound was prepared by a method analogous to that of Example 1 step (ix) using the product of step (ii). Yield 6 g.

MS: APCI (+ve): 529 (M+H)

### (iv) Methyl (3-{[[4-(6-amino-2-butoxy-8-oxo-7,8-dihydro-9H-purin-9-yl)butyl](3-hydroxypropyl)amino]methyl}phenyl)acetate

The title compound was prepared by a method analogous to that of Example 1 step (vii) using the product of step (iii). Yield 10 g.

MS: APCI (+ve): 515 (M+H)

### (v) Methyl [3-({[4-(6-amino-2-butoxy-8-oxo-7,8-dihydro-9H-purin-9-yl)butyl][3-(4-methylpiperazin-1-yl)propyl]amino}methyl)phenyl]acetate

The title compound was prepared by a method analogous to that of Example 1 step (x) using the product of step (iv). Yield 200 mg.

¹H NMR [symbol] (DMSO-d₆) 9.81 (1H, s), 7.27-7.03 (4H, m), 6.38 (2H, s), 4.18-4.08 (2H, m), 3.65-3.60 (4H, m), 3.58 (3H, s), 3.48-3.40 (2H, m), 3.37-3.26 (4H, m), 2.39-2.27 (4H, m), 2.27-2.19 (4H, m), 2.20-2.14 (2H, m), 2.10 (3H, s), 1.69-1.57 (4H, m), 1.55-1.43 (2H, m), 1.44-1.32 (4H, m), 0.91 (3H, t).

MS: APCI (+ve): 597 (M+1H)

### Biological Assays

### (1) Interferon-Inducing Activity of Rat Spleen Cells (In Vitro)

Spleens were removed from male Sprague-Dawley rats (approximately 8-10 weeks old) and a splenocyte suspension was prepared in serum-free MEM medium (modified Eagle's medium). Test compounds were dissolved in dimethylsulfoxide (DMSO), and incubated with splenocytes (5×10⁶ cells/ml) keeping the final DMSO concentration at 0.1%. Incubations were for 24 hours at 37° C. under an atmosphere of 5% carbon dioxide (CO₂) at which point supernatants were collected and analyzed for interferon alpha (IFNα). IFNα levels were determined in a bioassay by measuring the IFNα-mediated inhibition of vesicular stomatitis virus-induced cellular death of L929 cells. Values quoted are for the log of the minimum effective concentration (MEC) of test compound required to induce IFNα.

| Example No. | 1 | 2 | 3 | 4 | 5 | 6 |
|---|---|---|---|---|---|---|
| Log MEC | 9.5 | 9.5 | 9.5 | 9.5 | 9.5 | 9.0 |

### (2) Human TLR7 Assay

Recombinant human TLR7 was stably expressed in a HEK293 cell line already stably expressing the pNiFty2-SEAP reporter plasmid; integration of the reporter gene was maintained by selection with the antibiotic zeocin. The most common variant sequence of human TLR7 (represented by the EMBL sequence AF240467) was cloned into the mammalian cell expression vector pUNO and transfected into this reporter cell-line. Transfectants with stable expression were selected using the antibiotic blasticidin. In this reporter cell-line, expression of secreted alkaline phosphatase (SEAP) is controlled by an NFkB/ELAM-1 composite promoter comprising five NFkB sites combined with the proximal ELAM-1 promoter. TLR signaling leads to the translocation of NFkB and activation of the promoter results in expression of the SEAP gene. TLR7-specific activation was assessed by determining the level of SEAP produced following overnight incubation of the cells at 37° C. with the standard compound in the presence of 0.1% (v/v) dimethylsulfoxide (DMSO). Concentration dependent induction of SEAP production by compounds was expressed as the minimal effective concentration of compound to induce SEAP release (pMEC).

Compound of Example 2: pMEC 7.4
Compound of Example 3: pMEC 7.7
Compound of Example 5: pMEC 7.2.

### Solubility Testing

Saturated solutions for determining the solubility were prepared by placing about 0.3-3.0 ml of 0.1 M phosphare buffer in glass screw-top sample tubes along with some of the test compound. The tubes were then shaken overnight at constant temperature (20° C.). After shaking, undissolved material should be present in the solution, and more test compound should be added and shaking continued if this is not the case. The samples were then transferred to a centrifuge tube and centrifuged using a Heraeus Biofuge Fresco centrifuge at 13000 rpm for 30 minutes. The supernatant was then removed, placed in a new centrifuge tube and centrifuged again for 30 minutes at 13000 rpm. The undissolved material formed a pellet at the bottom of the tube and the liquid above the pellet was removed and was ready for assaying. The solution was then analysed using HPLC with UV quantification. A standard was also prepared by accurately weighing a sample of the test compound and dissolving it in a suitable volume of a solvent that will dissolve it completely (typically, DMSO, ethanol or methanol). This sample was then analysed by HPLC/UV.

### Results

The solubility was calculated from the observed peak areas in the HPLC/UV chromatograms along with corrections for any dilutions of the sample and differences in injection volumes. The following equation was used:

$$Solubility ( mg \text{/} ml ) = ( \frac{\begin{matrix}Std Conc ( mg \text{/} ml ) \cdot Sample Peak Area \cdot \\ Sample Dilution factor \cdot Std Inj Vol\end{matrix}}{Std Peak Area \cdot Sample Inj Vol} )$$

| Example | 1 | 2 | 3 | 4 | 5 | 6 |
|---|---|---|---|---|---|---|
| Solubility (mg/ml) | 0.98 | 1.94 | 0.85 | 0.83 | 1.82 | 1.21 |

| Comparison Examples from PCT/JP2005/005401 | 2-13 | 2-31 | 2-35 |
|---|---|---|---|
| Solubility (mg/ml) | 0.05 | 0.02 | 0.16 |

1. A compound of formula 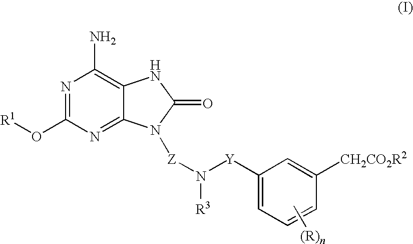
wherein
R¹ represents a C₁-C₆ alkyl group;
Z represents a C₂-C₆ alkylene group;
Y represents a C₁-C₃ alkylene group;
R² represents a C₁-C₆ alkyl group;
n is an integer from 0 to 2;
each group R independently represents halogen, C₁-C₃ alkyl, C₁-C₃ alkoxy or C₁-C₃ haloalkyl;
R³ represents —(CH₂)ₘ—NR⁴R⁵;
m is an integer from 2 to 6;
either R⁴ and R⁵ each independently represent hydrogen or C₁-C₆ alkyl, or R⁴ and R⁵ together with the nitrogen atom to which they are attached form a 3- to 8-membered saturated heterocyclic ring optionally comprising a further ring hetero group NR⁶; and
R⁶ represents hydrogen or C₁-C₆ alkyl;
or a pharmaceutically acceptable salt thereof.

2: The compound according to claim 1, wherein R¹ represents a C₁-C₄ alkyl group.

3: The compound according to claim 1, wherein Z represents a C₂-C₄ alkylene group.

4: The compound according to claim 1, wherein Y represents methylene.

5: The compound according to claim 1, wherein R⁴ and R⁵ each independently represent hydrogen or C₁-C₃ alkyl.

6: The compound according to claim 1, wherein R⁴ and R⁵ together with the nitrogen atom to which they are attached form a 5- to 6-membered saturated heterocyclic ring optionally comprising a further ring hetero group NR⁶.

7: The compound according to claim 1, wherein R² represents a C₁-C₃ alkyl group.

8: The compound according to claim 1 wherein
R¹ represents n-butyl;
Z represents n-propylene or n-butylene;
Y represents methylene;
R² represents methyl;
n is 0;
R³ represents —(CH₂)ₘ—NR⁴R⁵;
m is 2 or 3;
either R⁴ and R⁵ each independently represent hydrogen or methyl, or R⁴ and R⁵ together with the nitrogen atom to which they are attached form a 5- to 6-membered saturated heterocyclic ring optionally comprising a further ring hetero group NR⁶; and
R⁶ represents methyl.

9: The compound according to claim 1 selected from:
Methyl [3-({[3-(6-amino-2-butoxy-8-oxo-7,8-dihydro-9H-purin-9-yl)propyl][3-(dimethylamino)propyl]amino}methyl)phenyl]acetate,
Methyl (3-{[[3-(6-amino-2-butoxy-8-oxo-7,8-dihydro-9H-purin-9-yl)propyl](3-pyrrolidin-1-ylpropyl)amino]methyl}phenyl)acetate,
Methyl [3-({[3-(6-amino-2-butoxy-8-oxo-7,8-dihydro-9H-purin-9-yl)propyl][3-(4-methylpiperazin-1-yl)propyl]amino}methyl)phenyl]acetate,
Methyl [3-({[3-(6-amino-2-butoxy-8-oxo-7,8-dihydro-9H-purin-9-yl)propyl][2-(dimethylamino)ethyl]amino}methyl)phenyl]acetate,
Methyl [3-({[3-(6-amino-2-butoxy-8-oxo-7,8-dihydro-9H-purin-9-yl)propyl][3-(methylamino)propyl]amino}methyl)phenyl]acetate,
Methyl [3-({[4-(6-amino-2-butoxy-8-oxo-7,8-dihydro-9H-purin-9-yl)butyl][3-(4-methylpiperazin-1-yl)propyl]amino}methyl)phenyl]acetate,
and pharmaceutically acceptable salts of any one thereof.

10: A process for the preparation of a compound of formula (I) or a pharmaceutically acceptable salt thereof as defined in claim 1 which comprises,
(a) reacting a compound of formula 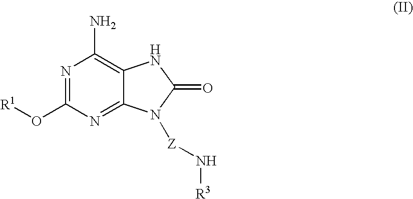
wherein Z, R¹ and R³ are as defined in formula (I), with a compound of formula 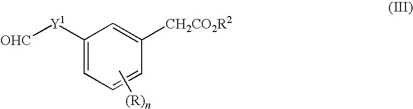
wherein Y¹ represents a bond or C₁-C₂ alkylene group and n, R and R² are as defined in formula (I) in the presence of a suitable reducing agent; or
(b) reacting a compound of formula (II) as defined in (a) above with a compound of formula 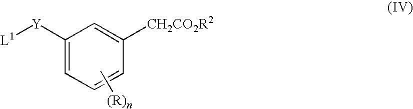
wherein L¹ represents a leaving group and n, Y, R and R² are as defined in formula (I) in the presence of a suitable base; or
(c) reacting a compound of formula 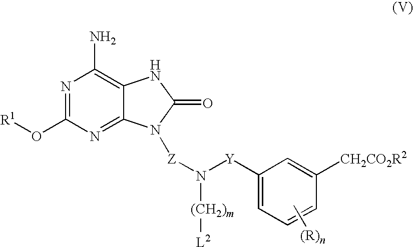
wherein L² represents a leaving group and m, n, Y, Z, R, R¹ and R² are as defined in formula (I), with a compound of formula (VI), HNR⁴R⁵, wherein R⁴ and R⁵ are as defined in formula (I); or
(d) reacting a compound of formula 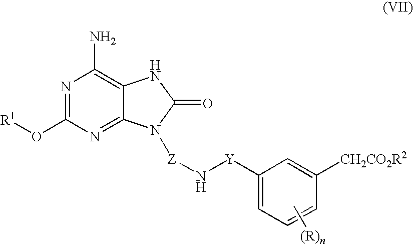
wherein n, Y, Z, R, R¹ and R² are as defined in formula (I), with a compound of formula
L³-(CH₂)ₘ—NR⁴R⁵ (VIII)
wherein L³ represents a leaving group and m, R⁴ and R⁵ are as defined in formula (I); or
(e) reacting a compound of formula (VII) as defined in (d) above with a compound of formula
OHC—(CH₂)ₘ₋₁—R⁴R⁵ (IX)
wherein m, R⁴ and R⁵ are as defined in formula (I) in the presence of a suitable reducing agent;
and optionally after (a), (b), (c), (d) or (e) carrying out one or more of the following:
converting the compound obtained to a further compound of formula (I)
forming a pharmaceutically acceptable salt of the compound.

11: A pharmaceutical composition comprising a compound of formula (I) or a pharmaceutically acceptable salt thereof as claimed in claim 1 or claim 9 in association with a pharmaceutically acceptable adjuvant, diluent or carrier.

12: A process for the preparation of a pharmaceutical composition as claimed in claim 11 which comprises mixing a compound of formula (I) or a pharmaceutically acceptable salt thereof as claimed in claim 1 or claim 9 with a pharmaceutically acceptable adjuvant, diluent or carrier.

13-16. (canceled)

17: A method of treating, or reducing the risk of, a disease or condition in which modulation of TLR7 activity is beneficial which comprises administering to a patient in need thereof a therapeutically effective amount of a compound of formula (I) or a pharmaceutically acceptable salt thereof as claimed in claim 1.

18: A method of treating, or reducing the risk of, an allergic or viral disease or cancer which comprises administering to a patient in need thereof a therapeutically effective amount of a compound of formula (I) or a pharmaceutically acceptable salt thereof as claimed in claim 1.

19: A method of treating, or reducing the risk of, an obstructive airways disease or condition which comprises administering to a patient in need thereof a therapeutically effective amount of a compound of formula (I) or a pharmaceutically acceptable salt thereof as claimed in claim 1.
